(19) United States
(12) Patent Application Publication
(10) Pub. No.: US 2007/0032736 A1
(43) Pub. Date: Feb. 8, 2007

(54) Method of predicting outcome of a stroke using eeg

(75) Inventors: Simon Peter Finnigan, St. Lucia (AU); Jonathan Brandon Chalk, St. Lucia (AU); Stephen Edward Rose, St. Lucia (AU)

Correspondence Address: NIXON & VANDERHYE, PC, 901 NORTH GLEBE ROAD, 11TH FLOOR, ARLINGTON, VA, 22203, US

(73) Assignee: THE UNIVERSITY OF QUEENSLAND, QUEENSLAND (AU)

(21) Appl. No.: 10/555,272

(22) PCT Filed: May 4, 2004
(86) PCT No.: PCT/AU04/00578

§ 371 Date: Nov. 2, 2005

(30) Foreign Application Priority Data
May 2, 2003 (AU) 2003902115

(51) Int. Cl. A61B 5/04

(52) U.S. Cl. 600/544

(57) ABSTRACT

EEG measures are used to predict neurological developments resulting from a stroke or similar cerebral ischaema in a person. EEG measures are acquired from the person at two time-points in an acute phase of the stroke, within 18 hours of onset of symptoms of stroke, with acquisition commencing within 7 hours of onset of the symptoms. The acquired EEG measures are processed to obtain a delta band power change measure, known as the acute delta change index (aDCI). Subsequent clinical outcome in the patient (e.g. at 30 days post-stroke) is predicted on the basis of the aDCI.

This invention relates to a method of predicting the evolution and clinical outcome of a stroke or similar ischaemic infarction, using EEG measures acquired in the acute phase of a stroke, i.e. obtained shortly after the onset of stroke symptoms.

### BACKGROUND ART

Typically, a person suffers an ischaemic infarction or stroke when a blood vessel is blocked, causing cerebral nervous tissue to be deprived of oxygen. In the initial few hours after a stroke, there is usually a significantly reduced blood supply to a region of nervous tissue due to a blocked or nearly-blocked blood vessel which would otherwise supply oxygen to that tissue. The nervous tissue deprived of adequate blood supply does not necessarily die immediately. It can often die over the next 18 hours or so. The prediction of the final size of the stroke (i.e. the final volume of dead tissue) and more saliently, patients' clinical outcomes, is very difficult.

A principal challenge in acute stroke therapy is to accurately identify, monitor, and predict the progression of stroke evolution. If these objectives can be achieved, the patient can receive optimal treatment. The efficacy of drugs can also be evaluated.

Existing methods of stroke evaluation are generally qualitative, and rely on the use of subjective measures such as operator defined regions of interest on diffusion and perfusion maps to enable prediction of infarct size. However, these methods are time consuming to implement and require highly skilled practitioners. Further, there is a limited time window of opportunity for the administration of thrombolytic or neuroprotective therapy.

The principal EEG indices of sub-acute (>24 hours) stroke and other ischaemic brain insults are well documented. The most common finding is a shift to a preponderance of high-voltage, slow delta (14 Hz) oscillations, often localised to EEG acquired from electrode sites overlying the ischaemic area. This increase in delta power (voltage squared) may sometimes be accompanied by a concomitant increase in theta (4-8 Hz) activity, and/or by a decrease in alpha (8-13 Hz) activity (particularly when the lesion encompasses occipital or parietal areas).

Quantitative EEG (qEEG) techniques include the computation of power and associated scalp topographic maps, for given frequency bands. Such techniques have been used during the past three decades to illustrate, diagnose, and investigate brain pathophysiology following stroke, and the efficacy of qEEG in this context has been well-demonstrated. For example, in the sub-acute post-stroke period qEEG topographic maps have been shown to indicate pathophysiological foci before they are detectable in computed tomography (CT) scans, and such foci have been demonstrated to reliably correlate with the location of the lesion as indicated by CT and MRI. In addition, it has recently been reported⁸ that qEEG variables, derived from data acquired within 72 hours of the stroke, can be employed to predict subsequent clinical outcomes following ischaemic stroke and further, that these variables possess higher prognostic value than the Canadian Neurological Scale in this context.

Previous investigations have typically collected a maximum of twenty minutes of EEG data at some point within the first 24 to 36 hours or within the first 3 to 4 days post-stroke. However, qEEG data has not been employed to systematically monitor brain pathophysiology over the course of hours, commencing in the hyper-acute (<7 hours) post-stroke phase.

Neuroimaging techniques, based on the use of diffusion and perfusion weighted MRI have been shown to be extremely useful for the identification of ischemic tissue and more importantly, for predicting functional outcome. International patent application no. PCT/AU02/00256 describes a method for predicting infarct evolution using magnetic resonance imaging (MRI) and image processing.

A number of measures derived from magnetic resonance imaging (MRI) data have previously been shown to generally have at least a modest correlation with clinical outcome. Using diffusion- (DWI) and perfusion-weighted (PWI) MRI scans acquired in acute stroke the volume of the DWI lesion or of the ischemic territory delineated on PWI mean transit time (MTT) maps has been shown to correlate with National Institutes of Health Stroke Scale (NIHSS) or Canadian Neurological Scale (CaNS) scores¹⁻⁴. It has also been demonstrated that these MRI measures can be mathematically modelled to predict final lesion volume^{5,6} but the latter often does not necessarily correlate well with clinical outcome⁴.

However due to a host of clinical, logistical and economic issues, MRI is not particularly practicable for continuous systematic investigations of the development of brain pathophysiology over the course of hours in the crucial acute post-stroke period (wherein current thrombolytic drugs, for example, must be administered).

It is an aim of this invention to provide an improved and potentially widely-utilisable method of predicting the evolution and functional outcome of a stroke or similar ischaemic infarction, using EEG measures.

### SUMMARY OF THE INVENTION

This invention provides a method of predicting neurological developments resulting from a cerebral disorder in a patient, comprising the steps of acquiring EEG measures from the patient at at least two time-points, processing the acquired EEG measures to obtain a delta band power measure at each of the two time-points, and predicting clinical status of the patient from the change in the delta band power measure between the two time-points.

In one embodiment in which the cerebral disorder is a stroke, the two time-points at which the EEG measures are acquired are in the acute post-stroke period. The acquired EEG measures are processed to obtain a delta band power change measure, known as the acute delta change index (aDCI). Subsequent clinical outcome in the patient (e.g. at 30 days post-stroke) is predicted on the basis of the aDCI and the patients' clinical status on hospital admission.

A decrease in the mean delta band power across all scalp electrodes, and thus a negative aDCI, indicates likely clinical improvement in the patient, and vice versa.

Preferably, the EEG data used to calculate the aDCI are acquired from the patient within approximately 18 hours, and commencing within 7 hours, of the onset of stroke-associated symptoms in the patient. Such data should be acquired from at least 20 electrodes distributed evenly across the scalp of the patient (and delta power and the aDCI will generally be greatest in EEG data from electrodes placed on regions of the scalp overlying the stroke).

The acquired EEG data are suitably processed, e.g. with appropriate computerised algorithms, to obtain a power spectrum in the delta band, which for the present purposes, is 1-4 Hz. The power spectrum is computed by a Fast Fourier Transform on artefact-free portions of EEG data. The aDCI, indexing both the direction and mean proportional change per hour in average scalp delta power, can then be used to predict clinical outcome for the stroke patient.

A preferred embodiment comprises the following steps:

1. Multi-channel EEG data is acquired within the acute phase of stroke (and commencing in the hyper-acute post-stroke period) using scalp electrodes, and artifact free periods of EEG data are selected.

2. The artifact free EEG data is bandpass filtered, separated into contiguous segments, and frequency band power is calculated for each electrode at a series of frequency points over a frequency range using the Fast Fourier Transform.

3. An “average scalp power spectrum” is computed by calculating the mean power (at each frequency point) across all scalp electrodes.

4. The frequency at which peak power occurs is determined in each patient's average scalp power spectrum and is verified in power spectra from several electrodes overlying the location of the stroke in the brain. This frequency is in the delta band (1-4 Hz), but varies from patient to patient. The power associated with this peak frequency is determined for each portion of artifact-free EEG data. Alternatively, delta power can be computed over the frequency interval between 1 Hz below peak delta power [but not below 1 Hz as the lower limit due to low-frequency EEG artifacts] and 1 Hz above peak delta power.

5. This delta power metric in data from one time-point is then subtracted from the same metric obtained at the next such time-point, and this difference score is divided by the time (in hours) that had elapsed between these two time-points in order to calculate the slope of a line constituting the cross-temporal change in delta power. The recommended minimum elapsed time between these two delta power metrics should generally be 3 hours. The resulting slope value is then converted to a quotient of the original delta power metric, producing the aDCI.

Reduction in delta power as recorded on EEG can provide an indication of subsequent clinical improvement in stroke patients over the following hours and up to at least 30 days post-stroke.

More sensitive measures of EEG coherence (both linear and non-linear) may also be used to predict clinical and functional outcomes from stroke.

A combination of EEG delta changes (and/or other EEG metrics), combined with MRI data obtained from the same patient at the same time, will likely improve prediction of stroke outcome.

The method of this invention has several advantages. First, the derivatives of data acquired from acute stroke patients allow prediction of the effects and efficacy of putative neuroprotective and thrombolytic agents, thereby enhancing the efficacy of the clinical management of acute stroke patients.

Given the unparalleled millisecond-scale temporal resolution of EEG in the monitoring of dynamic brain function, determination of the qEEG correlates of, for example, substantial recovery following re-perfusion in the acute post-stroke period, significantly enhances acute treatment decisions relating to the administration of appropriate pharmaceutical interventions and assists in assessing the efficacy of such interventions.

Furthermore, the identification of cross-temporal qEEG correlates of brain pathophysiology in the acute post-stroke period can be employed to generate an electrophysiological predictive model of stroke evolution and functional outcome.

Secondly, EEG data can be continuously collected from patients whilst in their hospital beds (including in emergency wards or intensive care units), and this can be acquired relatively readily due to low relative cost and rather widespread availability of EEG equipment.

These factors place the methodology of this invention in an optimal position to monitor acute post-stoke brain pathophysiology, and to yield indices which can be employed to effectively predict stroke evolution and to acutely assess the efficacy of pharmaceutical interventions.

Although the method is particularly suitable for predicting neurological developments resulting from a stroke or like cerebral ischaemia, it could be likewise applied to prediction of subsequent clinical status in conditions such as coma, brain haemorrhage, traumatic brain injury, and brain tumour.

In order that the invention may be more fully understood and put into practice, an example thereof will now be described, with reference to the accompanying drawings.

### BRIEF DESCRIPTION OF THE DRAWINGS

FIG. 1 illustrates EEG and MRI data from a patient who made an excellent early recovery. A and B: axial and left lateral EEG scalp Delta Power Maps (DPM) acquired 6.5 hours after onset of symptoms; C: initial DWI (6 hours); D: initial MTT map; E and F: axial and lateral DPM at 13 hours; G: 15 hour DWI scan and H: 30 day T2 MRI.

FIG. 2 illustrates EEG and MRI data from a patient who died at 12 days post-stroke. A and B: axial and lateral DPM acquired 9 hours after onset of symptoms; C: initial DWI (6 hours); D: initial MTT map; E and F: axial and right lateral DPM at 17 hours; G: 15 hour DWI scan and H: 15 hour T2 MRI.

FIG. 3 illustrates EEG and MRI data from a patient who received recombinant tissue Plasminogen Activator. A and B: axial and left lateral DPM acquired 6 hours after onset of symptoms; C: initial DWI (4.5 hours); D: initial MTT map; E and F: axial and lateral DPM at 12 hours; G: 13 hour DWI scan and H: 30 day T2 MRI.

FIG. 4 illustrates the strong, positive correlation (r=0.94 across the 12 patients whose data is plotted) between acute qEEG changes (the acute delta change index: aDCI) and patients' clinical outcomes (indexed by the NIHSS score at 30 days poststroke).

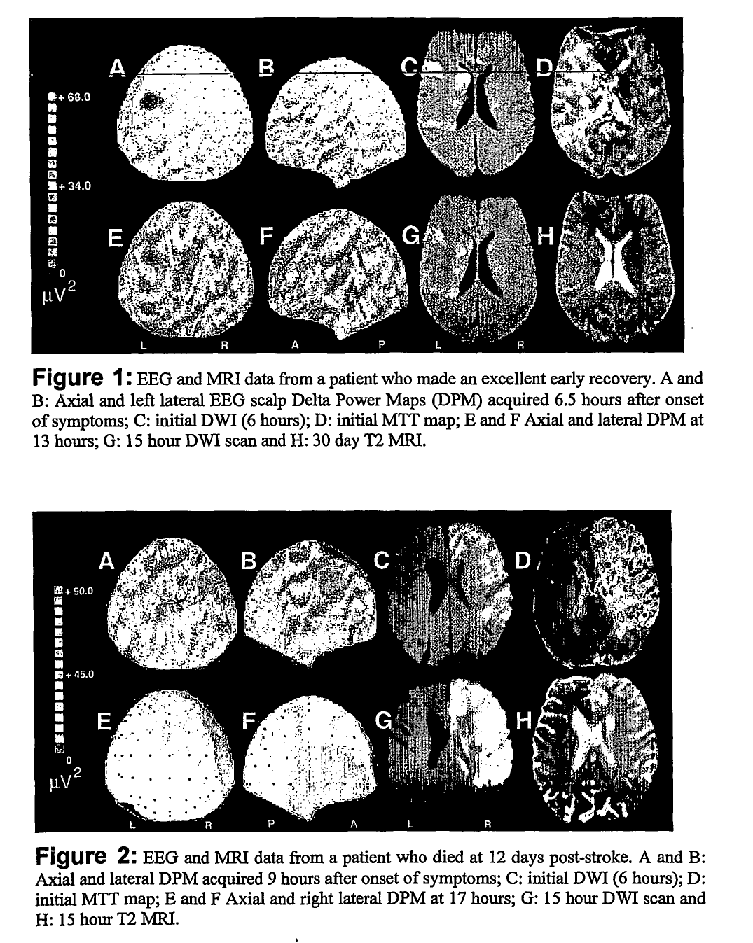

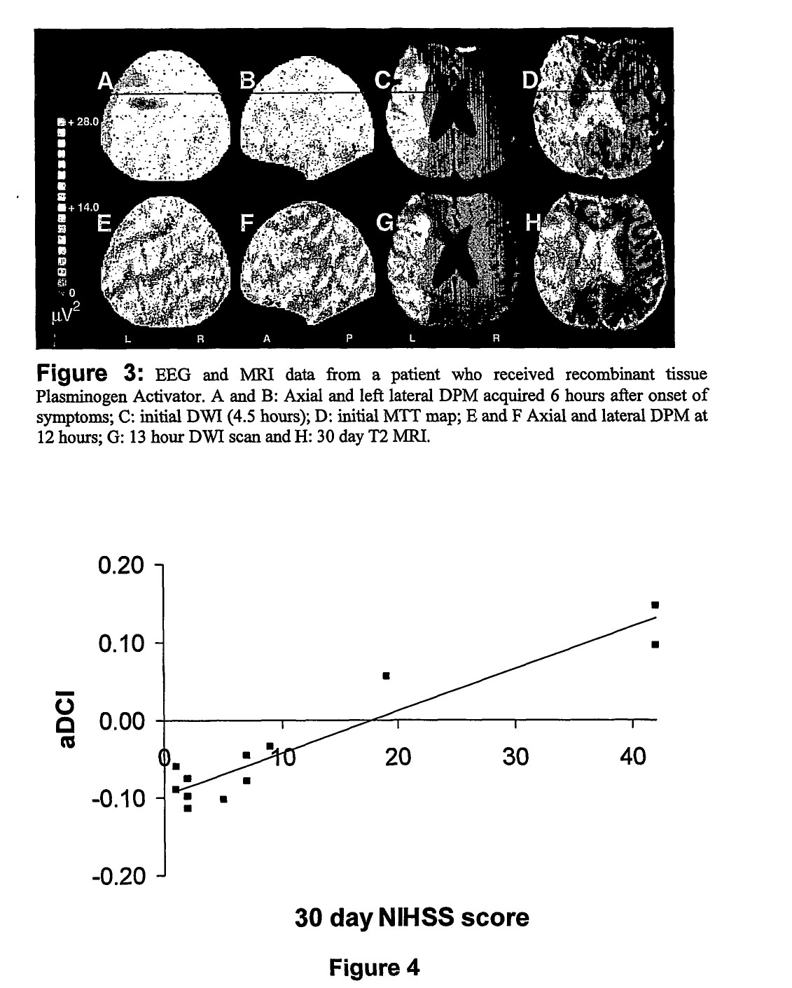

### DESCRIPTION OF AN EXAMPLE OF INVENTION

To demonstrate that qEEG measures acquired in the acute phase of stroke can be used to predict stroke evolution and functional outcome (particularly compared to existing established techniques utilising diffusion and perfusion weighted MRI), serial qEEG measures were recorded from a sample of eleven acute stroke patients and correlated with clinical outcome as indexed by the National Institutes of Health Stroke Scale (NIHSS) score. In addition, the relationship between these measures and the volumes of the acute DWI and PWI MRI lesions, were also investigated.⁹

Clinical Data from Stroke Patients

The NIHSS score was used to evaluate neurological impairment from stroke. The scale is an 11-item, clinical evaluation instrument widely used in clinical trials and practice, the reliability and validity of which is widely documented. The scale was administered on admission and at 24±2 hours, 48±2 hours, 72±2 hours, and 30±2 days, post-stroke.

EEG Data Acquisition and Analysis

Following the first MRI scanning session, patients were taken to their beds in a hospital ward. An elastic cap (Quik-Cap, Neuromedical Supplies) in which were embedded 62 sintered Ag/AgCl scalp electrodes, was fitted to each patient's head. Electrode locations corresponded to the following sites of the International 10-20 system: FPz, FP1, FP2, AF3, AF4, AF7, AF8, Fz, F1, F2, F3, F4, F5, F6, F7, F8, FCz, FC1, FC2, FC3, FC4, FC5, FC6, FT7, FT8, Cz, C1, C2, C3, C4, C5, C6, T7, T8, CPz, CP1, CP2, CP3, CP4, CP5, CP6, TP7, TP8, Pz, P1, P2, P3, P4, P5, P6, P7, P8, POz, PO3, PO4, PO5, PO6, PO7, PO8, Oz, O1, O2. The use of electrode caps is not essential, nor is such a high-density electrode montage. Twenty is the recommended minimum number of scalp electrodes

Vertical eye movements and blinks were monitored via two electrodes, one placed on the supraorbital ridge of, and one below, the left eye. Horizontal eye movements were monitored via two electrodes, one on the outer canthus of each eye. At acquisition, all electrodes' signals were referenced to a linked pair of electrodes, one positioned on each mastoid process. All electrodes were filled with conducting gel prior to data acquisition. Electrode impedances were predominantly 10-20 kΩ or less. EEG data was filtered (bandpass; 0.01-100 Hz) online and digitised at a sampling rate of 500 Hz.

Recordings were made using a Neuroscan SynAmps 64 channel digital EEG amplification and acquisition system. EEG was acquired continuously from the earliest practicable time post-MRI scan (approximately 7±2 hours post-stroke) until 15±2 hours post-stroke. At least several minutes of artefact-free EEG data was acquired within both the first and last hours of recording, during which times the patient was awake but resting quietly and still with eyes closed, with zero or minimal ambient noise and other activity in the room or immediate vicinity. In addition, between 20 and 30 minutes of EEG data were acquired under those conditions at 48±2 hours, and 30±2 days, post-stroke.

Three minutes of continuous artefact-free EEG data was filtered (bandpass; 0.2-40 Hz; 24 dB/octave), separated into contiguous segments each comprising 2048 data points, and EEG bandpower (representing voltage amplitude squared) was calculated for each electrode and at each 0.25 Hz point (over the range 0.5-40 Hz), using the Fast Fourier Transform.

In data from each time-point, an “average scalp power spectrum” was computed by calculating the mean power (at each frequency point) across all 62 scalp electrodes. The frequency at which peak power occurred, was determined in each patient's average scalp power spectrum. This frequency was always in the delta band (1-4 Hz), but varied between 1 and 1.75 Hz from patient to patient. The power associated with this peak frequency was determined for each portion of high-quality EEG data. (In alternative embodiments, instead of, or in addition to peak delta power, delta power in the range 14 Hz, and/or from 1 Hz below peak to 1 Hz above peak, may be used.)

To compute an acute delta change index (aDCI) reflecting the relative direction and rate of change of average scalp delta power across the acute post-stroke period, this delta power metric in data from the first high-quality EEG data time-point was subtracted from the same metric obtained at the second such time-point. The difference score was then divided by the time (in hours) that had elapsed between these two time-points in order to calculate the slope of a line constituting the cross-temporal change in delta power. The resulting slope value was then converted to a quotient of the original delta power metric, and this quotient was subsequently correlated with patients' clinical outcomes as indexed by the NIHSS score at 30 days post-stroke. That function was also computed as the change between time points one and two, divided by the elapsed time between these, and then by the original NIHSS score.

It was found that there is a strong relationship between reduction in delta power as recorded on EEG (the aDCI) and subsequent clinical outcome at 30 days in stroke patients as measured using clinical rating scales. As shown in FIG. 4, there is a strong correlation between acute changes in average scalp delta power (the aDCI), and functional outcome at 30 days post-stroke. Across the twelve subjects' data illustrated in FIG. 4, Pearson's correlation coefficient is highly significant at r=0.94 (out of a possible maximum of 1).

Thus, changes in delta power can be used to predict likely stroke development. In particular, the aDCI provides a quantitative measure of expected stroke outcome. Where drug therapy is applied, the efficacy of such therapy can be evaluated by reference to predicted functional outcome.

Calculation of the aDCI can be computerised or automated, using appropriate software.

The predictive approach may use regression, a statistical procedure that regularly follows that of correlation. A “line of best fit” or “regression line” is plotted to the data, using the Least Squares criterion. (This can be computed easily via any one of a number of standard computerised statistical packages). The subsequent NIHSS score at 30 days post-stroke (termed Y) can then be predicted on the basis of three variables:
1. The aDCI (termed X);
2. The slope of the regression line (i.e., the change in value on the Y-axis when X changes one unit; termed b); and
3. The intercept of the regression line (i.e., the predicted value of Y when X is zero; termed a).
   This is achieved via use of the following regression equation:
   Y=bX+a
   Predictive example based upon the twelve patients' data illustrated in FIG. 4:
Pearson's correlation coefficient, r=0.94
In this data set, the slope of the regression line, b=3.27; and the intercept, a =−0.05
A stroke patient presents with an initial NIHSSS of 25
EEG data is recorded from 7 hours to 13 hours post-stroke (with 3 minutes of high-quality, artefact-free data acquired at start and end)
Initial delta power at 7 hours (in the average scalp power spectrum) is 82.7, and at 13 hours is 26.5 (units are microvolts squared)
The slope of the “delta change” line is calculated as −9.37 (i.e. [26.5−82.7] divided by 6 [the number of hours elapsed])
The aDCI is calculated as −0.11 (i.e., the delta slope value, −9.37, divided by the initial delta power value of 82.7); this serves as the X-value for the regression equation
The predicted NIHSS score at 30 days, Y, is calculated using the abovementioned regression equation as −0.42 (i.e., 3.27 multiplied by −9.37, plus −0.05)
This NIHSS change represents the NIHSS score at the latter time-point minus the initial such score, and the difference divided by the initial score. Hence the predicted NIHSS at 30 days post-stroke would in this case be 14.5

The foregoing describes only one example of the invention, and modifications which are obvious to those skilled in the art may be made thereto without departing from the scope of the invention as defined in the accompanying claims.

For example, the general methodology of this invention may be applied to other disorders including cerebrovascular disorders (such as brain haemorrhages, various forms of hypoxia [disruption of regular oxygen supply to the brain], and severe migraines), coma states, and brain tumours, all of which elicit similar EEG outcomes to stroke (e.g., pronounced slowing of the brain's electrical oscillations, leading to high delta power), as well traumatic brain injuries and possibly mild traumatic brain injuries such as concussion. Related analysis & prognostic strategies (which might in future take into account EEG frequency bands other than delta) might be applied to other brain disorders such as mild cognitive impairment (a precursor of Alzheimers disease and other dementias) and epilepsy.

### REFERENCES

1. Schwamm L H, Koroshetz W J, Sorensen G, Wang B, Copen W A, Budzik R, Rordorf G, Buonanno F S, Schaefer P W, Gonzalez R G. Time course of lesion development in patients with acute stroke. Serial diffusion- and hemodynamic-weighted magnetic resonance imaging. Stroke. 1998; 29:2268-2276.
2. Barber P A, Darby D G, Desmond P M, Yang Q, Gerraty R P, Jolley D, Donnan G A, Tress B M, Davis S M. Prediction of stroke outcome with echoplanar perfusion- and diffusion-weighted MRI. Neurology. 1988; 51:418-426.
3. Beaulieu C, de Crespigny A, Tong D C, Moseley M E, Albers G W, Marks P M. Longitudinal magnetic resonance imaging study of perfusion and diffusion in Stroke: Evolution of lesion volume and correlation with clinical outcome. Ann Neurol. 1999; 46:568-578.
4. Baird A E, Lovblad K O, Dashe J F, Connor A, Burzynski C, Schlaug G, Straroselskaya, Edelman R R, Warach S. Clinical correlations of diffusion and perfusion lesion volumes in acute ischemic stroke. Cerebrovasc Dis. 2000; 10:441-44.
5. Mitsias P D, Jacobs M A, Hammound R, Pasnoor M, Santhakumar S, Papamitsakis N I H, Soltanian-Zadeh H, Lu M, Choop M, Patel S C. Multiparametric MRI ISODATA ischemic lesion analysis. Correlation with the clinical neurological deficit and single-parameter MRI techniques. Stroke. 2002; 33:2839-2844.
6. Wu O, Koroshetz W J, Ostergaard L, Buonanno F S, Copen W A, Gonzalez R G, Rordorf G, Rosen B R, Schwamm L H, Weisskoff R M, Sorensen A G. Stroke. 2001; 32:933-942.
7. Rose S E, Chalk J B, Griffin M, Janke A L, Chen F, McLachan G J, Peel D, Zelaya F O, Markus H S, Jones D K, Simmons A, O'Sullivan M, Jarosz J M, Strugnell W, Doddrell D M, Semple J. MRI based diffusion and perfusion predictive model to estimate stroke evolution. Magn. Reson. Imaging. 2001; 19:1043-1053.
8. Cuspineda E, Machado C, Aubert E, Galan L, Llopis F, Avila Y. Predicting outcome in an acute stroke: a comparison between QEEG and the Canadian Neurological Scale. Clin. Electroencephalogr. 2003; 34:1-4.

9. Finnigan S, Rose S., Walsh M, Griffin M, Janke A L, McMahon K L, Gillies R, Strudwick M W, Pettigrew C M, Semple J, Brown J, Brown P, Chalk J B. Correlation of quantitative EEG in acute stroke with 30 day NIHSS score: A comparison with diffusion and perfusion MRI. Stroke 2004; 35:899-903.

TABLE 1
Patient Demographics, Vascular Territories, Times of
MRI and EEG acquisition, and NIHSSS.

| | | | Acute Measurement | | | |
|---|---|---|---|---|---|---|
| | Age | Vascular | Time (hrs) | | NIHSSS | |
| Patient | Y/sex | Territory | MRI | EEG | Initial | 30 d |
| 1 | 83/F | RPCA | 3.5 | 5.5 | 14 | 5 |
| 2 | 57/F | LMCA | 5.0 | 6.5 | 7 | 2 |
| 3* | 83/F | RMCA | 7.0 | 8.0 | 22 | NA |
| 4 | 55/M | RICA | 3.0 | 5.0 | 5 | 2 |
| 5 | 87/F | RMCA | 5.5 | 7.5 | 19 | 9 |
| 6 | 64/M | LMCA | 4.5 | 6.0 | 4 | 1 |
| 7 | 85/M | LMCA | 5.5 | 7.5 | 25 | 7 |
| 8⁼ | 67/M | LMCA | 4.5 | 6.0 | 21 | 7 |
| 9* | 86/F | LMCA | 4.5 | 6.3 | 33 | NA |
| 10 | 86/F | LPCA | 5.0 | 6.5 | 18 | 1 |
| 11 | 66/M | LMCA | 7.0 | 8.0 | 11 | 19 |

*Patient died before 30-day follow up scan,
⁼Patient received tissue Plasminogen Activator after initial MRI scan.
L Left R Right MCA, middle cerebral artery,
PCA, posterior cerebral artery,
ICA, internal carotid artery.

TABLE 2
MRI and EEG indices.

| | Lesion Volumes (ml) | | | | qEEG |
|---|---|---|---|---|---|
| Patient | DWI 5 hr | MTT 5 hr | DWI 15 hr | T2 30 d | aDCI^{‡} |

| | | | | | |
|---|---|---|---|---|---|
| 1 | 5.6 | 14.8 | 7.7 | 6.0 | −0.10 |
| 2 | 25.3 | 188.3 | 29.8 | 9.0 | −0.17 |
| 3* | 205.7 | 424.8 | 263.9 | NA | 0.15 |
| 4 | 26.6 | 50.4 | 29.5 | 22.9 | −0.10 |
| 5 | 32.8 | 108.2 | 46.9 | 46.3 | −0.04 |
| 6 | 11.1 | 98.1 | 12.4 | 7.4 | −0.06 |
| 7 | 35.7 | 228.5 | 46.4 | 37.0 | −0.08 |
| 8⁼ | 134.5 | 199.8 | 143.0 | 167.3 | −0.05 |
| 9* | 224.3 | 483.1 | 298.5 | NA | 0.10 |
| 10 | 5.1 | 14.6 | 9.1 | 0.9 | −0.09 |
| 11 | 5.1 | NA | 14.0 | 144.7 | 0.06 |

*Patient died before 30-day follow up scan,
⁼Patient received tissue Plasminogen Activator after initial MRI scan and EEG recording.
^{‡}qEEG aDCI (acute delta change index) represents the mean percentage scalp delta power change per hour.

1. A method of predicting neurological developments resulting from a cerebral disorder in a patient, comprising the steps of
acquiring EEG measures from the patient at at least two time-points,
processing the acquired EEG measures to obtain a delta band power measure at each of the two time-points, and
predicting clinical status of the patient from the change in the delta band power measure between the two time-points.

2. A method as claimed in claim 1, wherein the cerebral disorder is a stroke or like cerebral ischaema.

3. A method as claimed in claim 2, wherein the EEG measures are obtained in an acute phase of the stroke.

4. A method as claimed in claim 3, wherein the power measure is a power spectrum over a frequency range.

5. A method as claimed in claim 4, wherein the power spectrum is obtained by a Fast Fourier Transform of artefact-free portions of the acquired EEG data.

6. A method as claimed in claim 3, wherein the EEG measures are acquired within 18 hours, and commencing within 7 hours, of onset of symptoms of stroke in the patient.

7. A method as claimed in claim 6, wherein the EEG data is acquired from a plurality of electrodes distributed evenly on a portion of the scalp of the patient overlying the stroke.

8. A method as claimed in claim 1, wherein the processing step is performed by computer software.

9. A method as claimed in claim 1, when used in combination with MRI data obtained from the same patient at the same time as the EEG data.

10. A method of predicting functional outcome of a stroke in a patient, comprising the steps of:
acquiring multi-channel EEG data within an acute phase of the stroke from at least twenty scalp electrodes on the patient;
selecting artifact free periods of the acquired EEG data;
frequency filtering the artifact free EEG data;
separating the filtered data into contiguous segments;
calculating frequency band power for each electrode at a series of frequency points over a frequency range using the Fast Fourier Transform;
computing an average scalp power spectrum by calculating the mean power (at each frequency point) across all scalp electrodes;
determining the delta band frequency at which peak power occurs in each patient's average scalp power spectrum and the power measure associated with this peak frequency for each portion of artifact-free EEG data;
subtracting the power measure at one time-point from the corresponding power measure at a subsequent time-point, and dividing the difference by the elapsed time between the two time-points to thereby calculate the slope of a line constituting the cross-temporal change in delta power; and
converting the resulting value of the slope to a quotient of the first power measure; and
predicting stroke outcome from the quotient.

11. A method as claimed in claim 10, wherein reduction in the power measure between the two time points provides an indication of expected clinical improvement in the stroke patient subsequently.

12. A method of predicting neurological developments resulting from a stroke or like cerebral ischaema in a person, comprising the steps of
acquiring EEG data from the person in an acute phase of the stroke,
processing the acquired EEG data to obtain a measure of power in the delta band at at least two time-points, and
predicting neurological outcome in the patient from the stroke from the change in the power measure between the two time-points.

13. A method as claimed in claim 12, wherein the EEG data are acquired within 18 hours of onset of symptoms of stroke in the person.

14. A method as claimed in claim 13, wherein the acquisition of the EEG data commences within 7 hours of onset of symptoms of stroke in the person.

15. A method as claimed in claim 12, wherein the processing step is performed by computer software.
